(19) United States
(12) Patent Application Publication
(10) Pub. No.: US 2024/0180798 A1
(43) Pub. Date: Jun. 6, 2024

(54) Oral Care Compositions

(71) Applicant: Colgate-Palmolive Company, New York, NY (US)

(72) Inventors: Diana Liceth HENAO ADLEVANKIN, Martinsville, NJ (US); Jean DENIS, Union, NJ (US); Lauren EVANS, Highland Park, NJ (US); Courtney Nicole HARRIS, Piscataway, NJ (US); Robert D'AMBROGIO, Princeton, NJ (US); Gokul GOVINDARAJU, Somerville, NJ (US)

(73) Assignee: Colgate-Palmolive Company, New York, NY (US)

(21) Appl. No.: 18/513,925

(22) Filed: Nov. 20, 2023

Related U.S. Application Data
(60) Provisional application No. 63/427,124, filed on Nov. 22, 2022.

(51) Int. Cl. A61K 8/21; A61K 8/19; A61K 8/24; A61K 8/44; A61K 8/46; A61K 8/60; A61Q 11/00

(52) U.S. Cl. CPC A61K 8/21; A61K 8/19; A61K 8/24; A61K 8/44; A61K 8/442; A61K 8/466; A61K 8/602; A61Q 11/00; A61K 2800/30

(57) ABSTRACT

Aspects of the invention are directed to oral care compositions. In accordance with an aspect, provided is an oral care composition having a surfactant system comprising at least one of: (i) a non-sulfate based anionic surfactant and an amphoteric surfactant, or (ii) a nonionic surfactant and an amino acid surfactant; a stannous ion source; a nitrate ion source; and a phosphate source.

### CROSS-REFERENCE TO RELATED PATENT APPLICATIONS

This application claims priority to U.S. Appl. No. 63/427,124, titled Oral Care Compositions and filed Nov. 22, 2022, the content of which is incorporated herein by reference in its entirety for all purposes.

### BACKGROUND

Dental erosion involves demineralization and damage to the tooth structure due to acid attack from nonbacterial sources. Erosion typically occurs initially in the enamel and, if unchecked, may proceed to the underlying dentin.

Dental erosion may be caused by or exacerbated by dental plaque. Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries, tooth decay, and/or tooth demineralization can be caused or exacerbated by acid produced from the bacterial degradation of fermentable sugar.

Stannous ion sources, such as stannous fluoride and stannous chloride, are known for use in clinical dentistry with a history of therapeutic benefits over forty years, and can have use in reducing certain bacterial growth in the oral cavity. However, until recently, the popularity of stannous ion sources has been limited by the instability in aqueous solutions. The instability of stannous salts in water is primarily due to the reactivity of the stannous ion (Sn²⁺). Stannous salts readily hydrolyze at a pH of above 4, resulting in precipitation from solution. It has traditionally been thought that this formation of insoluble stannous salts results in a loss of therapeutic properties. One common way to overcome the stability problems that can be associated with stannous ions is to limit the amount of water in the composition to very low levels, or to use a dual phase system.

Formulation of oral care compositions comprising stannous fluoride or stannous chloride and potassium salts have been particularly challenging because of stability issues between these two ingredients. U.S. Pat. No. 6,464,963 discloses that attempts to include both stannous fluoride and potassium salts, such as potassium nitrate, in a single-phase oral composition is hampered by the formation of insoluble stannic salts and compounds such as Sn(OH)2 and SnO2. U.S. Pat. No. 6,464,963 solves this stability problem by resorting to dual-component composition in which one component comprises the potassium salt, along with sodium hydroxide to adjust the pH of the component to 8 to 11, and the second component comprises the stannous fluoride or other stannous salts.

Another ingredient that can be found in oral care compositions is sodium lauryl sulfate. Sodium lauryl sulfate can be useful as it may increase phase stability (e.g., reduces the likelihood of phase separation) of the oral care composition and generally does not impact the stability of the active ingredients in the oral care composition. In some cases, a surfactant substitution—adding another surfactant to replace sodium lauryl sulfate—has a negative impact on the taste or stability of the active ingredients. Moreover, microbiological stability of the formulation can be negatively impacted by the absence of sodium lauryl sulfate. There are also production benefits to having sodium lauryl sulfate in a given formulation. For example, by replacing sodium lauryl sulfate with other surfactants, the oral care composition may become overly aerated during production and it may be more difficult to clean the equipment after the production process.

There is an ongoing desire for improved oral care compositions.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions. In accordance with an aspect, provided is an oral care composition having a surfactant system comprising at least one of: (i) a non-sulfate based anionic surfactant and an amphoteric surfactant, or (ii) a nonionic surfactant and an amino acid surfactant; a stannous ion source; a nitrate ion source; and a phosphate source (e.g., a phosphate ion source).

According to another aspect, provided is an oral care composition comprising a surfactant system comprising a betaine surfactant and a taurate surfactant; a stannous ion source; a nitrate ion source; and a phosphate source (e.g., a phosphate ion source).

In accordance with a further aspect, provided is an oral care composition comprising a surfactant system comprising a glutamate surfactant and a glucoside surfactant; a stannous ion source; a nitrate ion source; and a phosphate source (e.g., a phosphate ion source).

According to another aspect, provided is an oral care composition comprising a surfactant system comprising a betaine surfactant, a taurate surfactant, and an amino acid surfactant; a stannous ion source; a nitrate ion source; and a phosphate source (e.g., a phosphate ion source).

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a fluoride ion source and a stannous ion source. If a particular oral care composition includes both a fluoride ion source and a stannous ion source, stannous fluoride will serve only as either a fluoride ion source or a stannous ion source—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions and, e.g., to oral care compositions providing enhanced oral care benefit. The oral care compositions disclosed herein may simultaneously provide superior oral health benefits, achieve desirable foaming characteristics, and enhanced phase stability. It was advantageously and unexpectedly discovered that certain oral care compositions having surfactant systems of particular combinations of surfactants can achieve highly desirable oral are health benefits while simultaneously achieving desirable phase stability, desirable foam characteristics, and minimizing or excluding sodium lauryl sulfate and/or sodium lauryl ether sulfate.

In accordance with an aspect, provided is an oral care composition having a surfactant system comprising at least one of: (i) a non-sulfate based anionic surfactant and an amphoteric surfactant, or (ii) a nonionic surfactant and an amino acid surfactant; a stannous ion source; a nitrate ion source; and a phosphate source.

According to another aspect, provided is an oral care composition comprising a surfactant system comprising a betaine surfactant and a taurate surfactant; a stannous ion source; a nitrate ion source; and a phosphate source. In some embodiments, the oral care composition comprises a surfactant system comprising a betaine surfactant, a taurate surfactant, and an amino acid surfactant; a stannous ion source; a nitrate ion source; and a phosphate source (e.g., a phosphate ion source).

In accordance with a further aspect, provided is an oral care composition comprising a surfactant system comprising a glutamate surfactant and a glucoside surfactant; a stannous ion source; a nitrate ion source; and a phosphate source.

The oral care compositions may be formulated to have improved phase stability, while utilizing a minimal amount or no sodium lauryl sulfate and/or sodium lauryl ether sulfate. For example, the amount of sodium lauryl sulfate and/or sodium lauryl ether sulfate present in the oral care composition may be about 5 wt. % or less, about 4 wt. % or less, about 3 wt. % or less, about 2 wt. % or less, about 1 wt. % or less, about 0.5 wt. % or less, about 0.1 wt. % or less, or about 0.05 wt. % or less. In some embodiments, the oral care composition is substantially free of or free of sodium lauryl sulfate and/or sodium lauryl ether sulfate. In at least one embodiment, the oral care composition contains 0 wt. % or about 0 wt. % of sodium lauryl sulfate and/or sodium lauryl ether sulfate.

The oral care compositions may be in the form of dentifrice, toothpaste, mouthwash, and/or the like. Suitable components, such as those listed below, may be included or excluded from the formulations for the oral care compositions depending on the specific combination of other ingredients and the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions typically include a surfactant system comprising at least one of (i) a non-sulfate based anionic surfactant and an amphoteric surfactant and/or (ii) a nonionic surfactant and an amino acid surfactant. The surfactant system may, in some cases, comprise a non-sulfate based anionic surfactant, an amphoteric surfactant, and optionally an amino acid surfactant. In some embodiments, the surfactant system consists of (i) a non-sulfate based anionic surfactant and an amphoteric surfactant and/or (ii) a nonionic surfactant and an amino acid surfactant. For example, the surfactant system may consist of one or more non-sulfate based anionic surfactant and one or more amphoteric surfactant. By way of further example, the surfactant system may consist of one or more nonionic surfactant and one or more amino acid surfactant. By way of a further example, the surfactant system may consists of one or more non-sulfate based anionic surfactant, one or more amphoteric surfactant, and one or more amino acid surfactant. In yet a further example, the surfactant system may consists of one or more non-sulfate based anionic surfactant, one or more amphoteric surfactant, and one or more nonionic surfactant.

As noted above, it was advantageously and unexpectedly discovered that certain oral care compositions having surfactant systems of certain combinations of surfactants can simultaneously achieve various benefits, such as highly desirable oral are health benefits, desirable stability, and desirable foam characteristics, while simultaneously maintaining and minimizing or excluding sodium lauryl sulfate and sodium lauryl ether sulfate. The surfactant system may comprise 2 or more surfactants, e.g., 3, 4, 5, 6, 7, 8, 9, 10 surfactants, or a range formed therefrom. For example, the oral care composition may comprise 2 to 6, 2 to 5, 2 to 4, 2 or 3; from 3 to 6, or 3 or 4 surfactants.

The surfactant system may be present in the oral care composition in an amount from about 0.5 to about 9 wt. %, based on the total weight of the oral care composition. For example, the oral care composition may include a surfactant system in an amount from about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 2 to about 9 wt. %, about 2 to about 8 wt. %, about 2 to about 7 wt. %, about 2 to about 6 wt. %, about 2 to about 5 wt. %, about 2 to about 4 wt. %, about 2 to about 3 wt. %; from about 2.5 to about 9 wt. %, about 2.5 to about 8 wt. %, about 2.5 to about 7 wt. %, about 2.5 to about 6 wt. %, about 2.5 to about 5 wt. %, about 2.5 to about 4 wt. %, about 2.5 to about 3.5 wt. %; from about 3 to about 9 wt. %, about 3 to about 8 wt. %, about 3 to about 7 wt. %, about 3 to about 6 wt. %, about 3 to about 5 wt. %, about 3 to about 4 wt. %, about 3 to about 3.5 wt. %; from about 4 to about 9 wt. %, about 4 to about 8 wt. %, about 4 to about 7 wt. %, about 4 to about 6 wt. %, about 4 to about 5 wt. %; from about 5 to about 9 wt. %, about 5 to about 8 wt. %, about 5 to about 7 wt. %, about 5 to about 6 wt. %; from about 7 to about 9 wt. %, about 7 to about 8 wt. %, or any range or subrange thereof, based on the total weight of the oral care composition.

The oral care compositions typically comprise a surfactant system having one or more anionic surfactant(s). The surfactant system may include one or more anionic surfactant(s) in an amount that may range from about 0.1 to about 7 wt. %, based on the total weight of the oral care composition. In some instances, the one or more anionic surfactant(s) is present in the surfactant system in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the oral care composition.

The one or more anionic surfactant(s) may be selected from non-sulfate based anionic surfactants, such as sulfonated monoglycerides of fatty acids, isethionates, sarcosinates, taurate, or a combination of two or more thereof. Although the one or more anionic surfactant(s) are typically chosen from non-sulfate based anionic surfactants, the oral care composition may, in some embodiments, include a sulfate based anionic surfactants selected from ammonium lauryl sulfate, ammonium lauryl ether sulfate, sodium dodecyl sulfate, sodium coco-sulfate, ammonium coco-sulfate, and a combination of two or more thereof. For example, in some embodiments, the surfactant system includes a sulfate based anionic surfactant, wherein the oral care composition is substantially free of or free of sodium lauryl sulfate and/or sodium lauryl ether sulfate.

The anionic surfactant(s) may have at least one alkyl or acyl group, preferably, comprising a carbon chain of 8 to 21 carbons. In some instance, the alkyl group(s) of the anionic surfactant(s) comprise a carbon chain of 8 to 19 carbons, 8 to 17 carbons, 8 to 15 carbons, 8 to 13 carbons, 8 to 11 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof. The anionic surfactants disclosed herein may be incorporated in a salt form. The salt form of the anionic surfactants may have an alkali metal (e.g., sodium or potassium) and/or ammonium group.

Non-limiting examples of isethionate surfactants include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

The anionic surfactant may be selected from taurate surfactants having a structure according to the following formula:

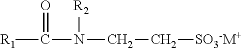

wherein R₁ is a saturated or unsaturated, straight or branched alkyl chain with 6 to 18 carbon atoms, R₂ is H or methyl, and M⁺ is H, sodium, or potassium (e.g., sodium methyl cocoyl taurate).

Additional examples of taurate surfactant salts include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations thereof. In some embodiments, the oral care composition comprises sodium lauroyl methyl taurate (or sodium methyl lauroyl taurate), sodium methyl cocoyl taurate (SMCT), or a combinations thereof. In at least one preferred embodiment, the surfactant system comprises sodium methyl cocoyl taurate. In some embodiments, one or more of the above anionic surfactants may be excluded from the oral care composition.

In some embodiments, the surfactant system preferably includes an amphoteric surfactant system in addition to an anionic surfactant. The surfactant system may comprise one or more amphoteric surfactant(s), e.g., in an amount from about 0.1 to about 5 wt. %, based on the total weight of the oral care composition. For example, the one or more amphoteric surfactant(s) may be present in the surfactant system in an amount from about 0.1 to about 4 wt. %, about 0.1 to about 3 wt. %, about 0.1 to about 2 wt. %, about 0.1 to about 1 wt. %; from about 0.3 to about 5 wt. %, about 0.3 to about 4 wt. %, about 0.3 to about 3 wt. %, about 0.3 to about 2 wt. %, about 0.3 to about 1 wt. %; from about 0.6 to about 5 wt. %, about 0.6 to about 4 wt. %, about 0.6 to about 3 wt. %, about 0.6 to about 2 wt. %, about 0.6 to about 1 wt. %; from about 1 to about 5 wt. %, about 1 to about 4 wt. %, about 1 to about 3 wt. %, about 1 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, including any range or subrange thereof, based on the total weight of the oral care composition.

Amphoteric surfactants are typically characterized by a combination of high surfactant activity, lather forming and mildness. The amphoteric surfactant may comprise a substituent containing 8 to 18 carbon atoms and a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched. In some cases, one of the aliphatic substituents of the amphoteric surfactant contains about 8 to about 18 carbon atoms and one of the aliphatic substituents contains an anionic water solubilizing group, e.g., carboxy, sulfonate, sulfate, phosphate, or phosphonate. The amphoteric surfactants disclosed herein may be present in the oral care composition in a salt form.

The amphoteric surfactants may include alkyl amphopropionates, betaines, alkyl sultaines, alkyl amphoacetates, or a combination of two or more thereof. Preferably, the oral care composition includes an amphoteric surfactant selected from betaine surfactants (also referred to herein as betaines). Examples of betaine surfactants include, e.g., alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine. In some instances, the betaine surfactant is selected from coca betaine, cocamidopropyl betaine, lauryl betaine, laurylhydroxy sulfobetaine, lauryldimethyl betaine, behenyl betaine, capryl/capramidopropyl betaine, lauryl hydroxysultaine, stearyl betaine, and a combination of two or more thereof. For instance, the betaine surfactant may be coco betaine, cocamidopropyl betaine, behenyl betaine, capryl/capramidopropyl betaine, and lauryl betaine, or a combination of two or more thereof. In at least one embodiment, the oral care composition comprises coco betaine, cocamidopropyl betaine, or a combination thereof. In some embodiments, one or more of the above amphoteric surfactants may be excluded from the oral care composition.

The oral care compositions may be formulated to have certain weight ratios of the amount of anionic surfactant(s) (e.g., taurate surfactant) to the amount of amphoteric surfactant(s) (e.g., betaine surfactant). For example, the oral care composition may have a weight ratio of the total amount of anionic surfactant(s) to the total amount of amphoteric surfactant(s) of from about 1:5 to about 5:1, about 1:4 to about 5:1, about 1:3 to about 5:1, about 1:2 to about 5:1, about 1:1 to about 5:1; from about 1:5 to about 4:1, about 1:4 to about 4:1, about 1:3 to about 4:1, about 1:2 to about 4:1, about 1:1 to about 4:1; from about 1:5 to about 3:1, about 1:4 to about 3:1, about 1:3 to about 3:1, about 1:2 to about 3:1, about 1:1 to about 3:1; from about 1:5 to about 2:1, about 1:4 to about 2:1, about 1:3 to about 2:1, about 1:2 to about 2:1, about 1:1 to about 2:1; from about 1:5 to about 1:1, about 1:4 to about 1:1, about 1:3 to about 1:1, about 1:2 to about 1:1, or about 1:1, including ranges or subranges formed thereof.

Additionally or alternatively, the surfactant system may comprise one or more of the amino acid derived surfactant(s) in an amount that may vary, but may be present in an amount from about 0.1 to about 7 wt. %, based on the total weight of the oral care composition. For example, the one or more amino acid derived surfactant(s) may be present in the surfactant system in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the oral care composition.

The amino acid derived surfactant may be anionic, non-ionic, amphoteric, or cationic. In some embodiments, the amino acid derived surfactant is an anionic surfactant. The amino acid derived surfactant may be based on alanine, arginine, aspartic acid, glutamic acid, glycine, isoleucine, leucine, lysine, phenylalanine, serine, tyrosine, valine, sarcosine, threonine, and taurine. The amino acid derived surfactants disclosed herein may be present in the oral care composition in a salt form. The most common cation associated with the acyl amino acid can be sodium or potassium. Alternatively, the cation can be an organic salt such as triethanolamine (TEA) or a metal salt.

The amino acid derived surfactant may be an acyl amino acid derived surfactant having an aliphatic carbon chain of 3 to 21 carbons. For instance, the amino acid derived surfactant may include an alkyl group comprised of a carbon chain of 3 to 21 carbons, 3 to 19 carbons, 3 to 17 carbons, 3 to 15 carbons, 3 to 13 carbons, 3 to 11 carbons, 3 to 9 carbons, 3 to 7 carbons; 4 to 21 carbons, 4 to 19 carbons, 4 to 17 carbons, 4 to 15 carbons, 4 to 13 carbons, 4 to 11 carbons, 4 to 9 carbons, 4 to 7 carbons; 6 to 21 carbons, 6 to 19 carbons, 6 to 17 carbons, 6 to 15 carbons, 6 to 13 carbons, 6 to 11 carbons, 6 to 9 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof.

Preferably, the amino acid derived surfactant comprises a glutamic acid derived surfactant. The glutamic acid derived surfactant may be selected from an acyl glutamic acid derived surfactants having an alkyl group comprised of a carbon chain having 3 to 21 carbons or any of the ranges for the carbon chain described above with respect to the amino acid derived surfactant. For instance, the carbon chain of the acyl glutamic acid derived surfactant may be 4 to 19 carbons, optionally 6 to 17 carbons, or optionally 9 to 13 carbons. In some preferred embodiments, the amino acid derived surfactant comprises sodium cocoyl glutamate. In some embodiments, one or more of the above amino acid derived surfactants may be excluded from the oral care composition.

In some embodiments, the surfactant system preferably includes a nonionic surfactant in addition to an amino acid based surfactant. The surfactant system may comprise one or more nonionic surfactant(s). The one or more nonionic surfactant(s) may be present in an amount from about 0.1 to about 7 wt. %, based on the total weight of the oral care composition. In some instances, the one or more nonionic surfactant(s) is present in the surfactant system in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the oral care composition.

The one or more nonionic surfactant(s) may be selected from glucosides, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethylene glycol octylphenol ethers, sorbitan alkyl esters, polyoxyethylene glycol sorbitan alkyl esters, or a combination of two or more thereof. In some embodiments, one or more of the above nonionic surfactants may be excluded from the oral care composition.

The oral care compositions may be formulated to have certain weight ratios of the amount of amino acid based surfactant(s) to the amount of nonionic surfactant(s). For example, the oral care composition may have a weight ratio of the total amount of amino acid based surfactant(s) to the total amount of nonionic surfactant(s) of from about 1:5 to about 5:1, about 1:4 to about 5:1, about 1:3 to about 5:1, about 1:2 to about 5:1, about 1:1 to about 5:1; from about 1:5 to about 4:1, about 1:4 to about 4:1, about 1:3 to about 4:1, about 1:2 to about 4:1, about 1:1 to about 4:1; from about 1:5 to about 3:1, about 1:4 to about 3:1, about 1:3 to about 3:1, about 1:2 to about 3:1, about 1:1 to about 3:1; from about 1:5 to about 2:1, about 1:4 to about 2:1, about 1:3 to about 2:1, about 1:2 to about 2:1, about 1:1 to about 2:1; from about 1:5 to about 1:1, about 1:4 to about 1:1, about 1:3 to about 1:1, about 1:2 to about 1:1, or about 1:1, including ranges or subranges formed thereof.

In addition to a surfactant system, the oral care compositions typically comprise a stannous ion source, preferably in an effective amount. The stannous ion source may be present in the oral care composition in an amount from about 0.1 to about 5 wt. %, based on the total weight of the oral care composition. For example, the amount of stannous ion present in the oral care composition may be from about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The stannous ion source may be selected from the group consisting of: stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and combinations of two or more thereof. The stannous source may comprise stannous fluoride, stannous chloride, stannous acetate, and a combination of two or more thereof. In at least one embodiment, the stannous ion source comprises stannous fluoride.

The oral care composition may include two or more, three or more, four or more, five or more, or six or more of stannous ion sources. For instance, oral care composition may comprise 2 to 7, 2 to 6, 2 to 5, 2 to 5, or 2 to 4; 3 to 7, 3 to 6, 2 to 5, or 3 to 5 stannous ion sources. In some embodiments, the oral care composition comprises stannous fluoride and stannous pyrophosphate.

Additionally or alternatively, the oral care composition may comprise stannous fluoride and stannous chloride.

The oral care compositions typically comprises one or more nitrate ion source, preferably present in an effective amount. The nitrate ion source(s) may be present in the oral care composition in an amount from about 0.1 to about 5 wt. %, based on the total weight of the oral care composition. In some instances, the amount of nitrate ion present in the oral care composition may be from about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The one or more nitrate ion source may be in the form of a salt or an ion derived therefrom, including, e.g., nitrate salt is selected from an alkali or alkaline earth metal nitrate. Examples of nitrate ion sources include lithium nitrate, sodium nitrate, potassium nitrate, magnesium nitrate, calcium nitrate, zinc nitrate, silver nitrate, ammonium nitrate, or a combination two or more thereof. In at least one embodiment, the nitrate ion source comprises potassium nitrate.

The oral care compositions may be formulated to have a molar ratio of nitrate ions to stannous ions, both measured as free ions, that is about 2:1 or less. For example, the oral care composition may have a molar ratio of nitrate ions to stannous ions, both measured as free ions, of from about 0.5:1 to about 2:1, about 0.5:1 to about 1.8:1, about 0.5:1 to about 1.6:1, about 0.5:1 to about 1.4:1, about 0.5:1 to about 1.2:1, about 0.5:1 to about 1:1; from about 0.7:1 to about 2:1, about 0.7:1 to about 1.8:1, about 0.7:1 to about 1.6:1, about 0.7:1 to about 1.4:1, about 0.7:1 to about 1.2:1, about 0.7:1 to about 1:1; from about 0.9:1 to about 2:1, about 0.9:1 to about 1.8:1, about 0.9:1 to about 1.6:1, about 0.9:1 to about 1.4:1, about 0.9:1 to about 1.2:1, about 0.9:1 to about 1:1, or any range or subrange thereof. In some embodiments, the oral care composition is formulated to have a molar ratio of nitrate ions to stannous ions, both measured as free ions, of about 1:1.

The oral care compositions typically comprises one or more phosphate source(s), e.g., a phosphate ion source(s). The phosphate source(s) is preferably present in an effective amount. In some instances, the oral care composition may include one or more phosphate source(s) in an amount from about 0.1 to about 7 wt. %, based on the total weight of the oral care composition. For example, the one or more phosphate source(s) may be present in the oral care composition in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the oral care composition.

The phosphate source may be a phosphate ion source. The phosphate ion source may preferably comprise a soluble phosphate compound, such as a soluble phosphate salt. The phosphate source may, in some cases, comprise an insoluble phosphate compound, such as an insoluble phosphate salt. Examples of phosphate sources include tetrasodium pyrophosphate, dicalcium orthophosphate dihydrate, dicalcium phosphate dihydrate, calcium hydrogen phosphate, calcium pyrophosphate, p-calcium pyrophosphate, tricalcium phosphate, calcium metaphosphate, potassium metaphosphate, sodium metaphosphate, or a combination thereof. The phosphate source may be chosen from orthophosphates, polymetaphosphates, pyrophosphates, and a combination of two or more thereof. In some instances, the phosphate source includes tetrasodium pyrophosphate (TSPP), sodium tripolyphosphate (STPP), or a combination thereof. In at least one embodiment, the phosphate source comprises tetrasodium pyrophosphate.

The oral care composition may include a zinc source (e.g., a zinc ion source). When present, the zinc source (e.g., zinc ion source) is preferably in an effective amount. The zinc source (e.g., zinc ion source) may be present in the oral care composition in an amount from about 0.1 to about 8 wt. %, based on the total weight of the oral care composition. For example, the amount of zinc source (e.g., zinc ion source) present in the oral care composition may be from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 8 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

The zinc source and/or zinc ion source may be in the form of a salt. For example, the zinc source may comprise one or more zinc salt(s) chosen from zinc sulfate, zinc chloride, zinc acetate, zinc phenol sulfonate, zinc borate, zinc bromide, zinc nitrate, zinc glycerophosphate, zinc benzoate, zinc carbonate, zinc carnosine, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, zinc lactate, zinc glycinate, zinc phosphate, and a combination of two or more thereof. In some embodiments, the zinc source is selected from zinc citrate, zinc oxide, zinc phosphate, zinc lactate, zinc sulfate, zinc silicate, zinc gluconate, and a combination of two or more thereof. In at least one embodiment, the zinc source consist of zinc citrate, zinc oxide, zinc phosphate, zinc lactate, zinc sulfate, zinc silicate, or a combination of two or more thereof.

The oral care composition may include two or more, three or more, four or more, five or more, or six or more zinc sources. For instance, oral care composition may comprise 2 to 7, 2 to 6, 2 to 5, 2 to 5, or 2 to 4; 3 to 7, 3 to 6, 2 to 5, or 3 to 5 zinc sources. In some embodiments, the oral care composition comprises zinc oxide, zinc citrate, zinc phosphate, or a combination of two or more thereof. In at least one preferred embodiment, the oral care composition comprises zinc phosphate.

The oral care compositions may be formulated to have a weight ratio of the amount of zinc oxide to zinc citrate of from about 1.5:1 to about 4.5:1. For instance, the weight ratio of the amount of zinc oxide to zinc citrate may be from about 1.5:1 to about 4.5:1, about 1.5:1 to about 4:1, about 1.5:1 to about 3.5:1, about 1.5:1 to about 3:1, about 1.5:1 to about 2.5:1; from about 2:1 to about 4.5:1, about 2:1 to about 4:1, about 2:1 to about 3.5:1, about 2:1 to about 3:1; from about 2.5:1 to about 4.5:1, about 2.5:1 to about 4:1, about 2.5:1 to about 3.5:1; from about 3:1 to about 4.5:1, about 3:1 to about 4:1, or any range or subrange formed thereof. In some embodiments, the weight ratio of the amount of zinc oxide to zinc citrate is about 2:1, about 2.5:1, about 3:1, about 3.5:1, about 4:1, or a range formed thereof.

The oral care compositions may include a fluoride ion source. The fluoride ion source may be present in an effective amount. In some cases, the fluoride ion source is present in the oral care composition in an amount of about 0.01 to about 5 wt. %, based on the total weight of the oral care composition. For example, the fluoride ion source is present in an amount of about 0.01 to about 5 wt. %, about 0.01 to about 4 wt. %, about 0.01 to about 3 wt. %, about 0.01 to about 2 wt. %, about 0.01 to about 1 wt. %; from about 0.05 to about 5 wt. %, about 0.05 to about 4 wt. %, about 0.05 to about 3 wt. %, about 0.05 to about 2 wt. %, about 0.05 to about 1 wt. %; from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion source are disclosed in U.S. Pat. Nos. 3,535,421, 4,885,155, and 3,678,154, the disclosure of each of which is hereby incorporated by reference in their entirety.

In certain embodiments, the oral care composition of the disclosure may contain stannous fluoride and a source of fluoride ions or fluorine-providing agents in amounts sufficient to supply, in total, from 25 ppm to 25,000 ppm (mass fraction) of fluoride ions, generally at least 500 ppm, e.g., from about 500 to about 2000 ppm, from about 800 to about 1800 ppm, from about 1000 to about 1600 ppm, from about 1200 to about 1550 ppm, or about 1450 ppm.

The oral care compositions may include one or more abrasive(s), e.g., in an amount from about 5 to about 35 wt. %, based on the total weight of the oral care composition. In some embodiments, the one or more abrasive(s) is present in an amount from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the oral care composition.

The one or more abrasive(s) may include: silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, urea-formaldehyde resin, polycarbonate, silicon carbide, boron carbide, microcrystalline wax, microcrystalline cellulose, including combinations of colloidal microcrystalline cellulose and carboxymethylcellulose, and combinations and derivatives thereof. For example, the one or more abrasive may be selected from silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, resinous abrasive materials such as particulate condensation products of urea and formaldehyde, and combinations thereof.

As used herein, “mica” refers to any of a group of hydrous aluminum silicate minerals with plate morphology and/or perfect basal (micaceous) cleavage. Mica can be, for example, sheet mica, scrap mica or flake mica, as exemplified by muscovite, biotite or phlogopite type micas. The abrasive may be selected from insoluble phosphates, such as orthophosphates, polymetaphosphates, pyrophosphates, and a combination thereof. Synthetic silicas include both silica gels and precipitated silicas that are prepared by the neutralization of aqueous silicate solutions with a strong mineral acid. Abrasives comprising silica may be useful in certain embodiments of the oral care composition. In certain embodiments, the oral compositions may comprise a particularly efficacious combination of silica abrasive particle species. For example, the abrasive(s) be selected from high cleaning silica.

The oral care compositions may include an abrasive system comprising two or more abrasives. For example, an abrasive system comprise 2 to 7, 2 to 6, 2 to 5, 2 to 5, or 2 to 4; 3 to 7, 3 to 6, 2 to 5, or 3 to 5 abrasives. The abrasives may comprise one or more cleaning abrasive and/or one or more polishing abrasives. As appreciated by one of skill in the art, a single abrasive species typically performs at least some cleaning and polishing simultaneously. However, particles are generally categorized in the art by the predominant effect they have on a target oral surface. Typically, “polishing abrasives” are considered to be relatively small particles having high hardness, where abrasives with relatively large particle sizes and low hardness are considered to be “cleaning abrasives.” In certain embodiments, the oral care composition comprises two or more abrasives comprising silica. In some embodiments, the first abrasive is selected to be a harder and smaller abrasive, e.g., a higher cleaning and/or polishing abrasive, and the second abrasive is a typical cleaning abrasive. In some embodiments, the oral care composition includes at least one polishing abrasive and/or at least one cleaning abrasive. Further examples of abrasives are disclosed in U.S. Patent Publication No. 2007/140986, which is incorporated herein in its entirety by reference for all purposes.

The oral care composition may include one or more thickening agent(s), e.g., in an amount from about 0.5 to about 20 wt. %, based on the total weight of the oral care composition. For example, the amount of thickening agent(s) in the oral care composition may be from about 0.5 to about 17 wt. %, about 0.5 to about 14 wt. %, about 0.5 to about 11 wt. %, about 0.5 to about 9 wt. %, about 0.5 to about 7 wt. %, about 0.5 to about 5 wt. %, about 0.5 to about 4 wt. %, about 0.5 to about 3 wt. %; from about 1 to about 20 wt. %, about 1 to about 17 wt. %, about 1 to about 14 wt. %, about 1 to about 11 wt. %, about 1 to about 9 wt. %, about 1 to about 7 wt. %, about 1 to about 5 wt. %, about 1 to about 4 wt. %, about 1 to about 3 wt. %; from about 2 to about 20 wt. %, about 2 to about 17 wt. %, about 2 to about 14 wt. %, about 2 to about 11 wt. %, about 2 to about 9 wt. %, about 2 to about 7 wt. %, about 2 to about 5 wt. %, about 2 to about 4 wt. %, about 2 to about 3 wt. %; from about 4 to about 20 wt. %, about 4 to about 17 wt. %, about 4 to about 14 wt. %, about 4 to about 11 wt. %, about 4 to about 9 wt. %, about 4 to about 7 wt. %; from about 7 to about 20 wt. %, about 7 to about 17 wt. %, about 7 to about 14 wt. %, about 7 to about 11 wt. %; from about 10 to about 20 wt. %, about 10 to about 17 wt. %, about 10 to about 14 wt. %; from about 14 to about 20 wt. %, about 14 to about 17 wt. %, or any range or subrange thereof, based on the total weight of the oral care composition.

Thickening agents may be referred to as “thickeners” or “viscosity modifying agents.” Thickening agents are typically included to increase the viscosity of the oral care compositions. Nonetheless, in some instances, certain thickening agents provide additional, surprising benefits to the oral care compositions.

The one or more thickening agent(s) may be selected from a polysaccharide, a silica thickener, an acrylate, a polymer, and a combination of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan, or a combination of two more thereof. Natural gums such as karaya, gum arabic, and gum tragacanth can also be incorporated. Colloidal magnesium aluminum silicate can also be used as a component of the thickening composition to further improve the composition's texture. Silica thickeners, which form polymeric structures or gels in aqueous media, may be present. Silica thickeners are typically physically and functionally distinct from the particulate silica abrasives as the silica thickeners are very finely divided and provide little or no abrasive action.

In some embodiments, the one or more thickening agent(s) comprises a polymer selected from polysaccharides, acrylates, polyvinyl pyrrolidone, polyitaconates, an acrylamides, and combinations thereof. Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000, preferably in the range 5,000 to 50,000. Polyvinyl pyrrolidones that have average molecular weights of 10,000, 30,000 and 40,000 may be commercially available from Sigma Chemical Co., GAF Corporation and Sigma Chemical Co. The polyvinyl pyrrolidone may form a hydrogen peroxide-polyvinyl pyrrolidone polymer complexes. Examples of polyvinyl pyrrolidone complexes include those disclosed in U.S. Pat. No. 5,122,370, the contents of which are incorporated herein by reference. In some embodiments, the polymer comprises crosslinked polyvinyl pyrrolidone (crosslinked PVP). In at least one embodiment, the polymer consists of polyvinyl pyrrolidone.

Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate, trifluoroethyl methacrylate, or a combination of two or more thereof.

Examples of acrylamides include, but are not limited to, acrylamide, methacrylamide and di(C₁-C₃₀) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide, 2-acrylamido-2-methylpropanesulfonic acid, or a combination of two or more thereof.

The oral care compositions may include one or more polyol(s). The one or more polyol(s) may be present in the oral care composition in an amount from about 20 to about 60 wt. %, based on the total weight of the oral care composition. For example, the oral care composition may include polyol(s) in an amount from about 20 to about 55 wt. %, about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 60 wt. %, about 25 to about 55 wt. %, about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 60 wt. %, about 28 to about 55 wt. %, about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 60 wt. %, about 31 to about 55 wt. %, about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 60 wt. %, about 37 to about 55 wt. %, about 37 to about 50 wt. %, about 37 to about 45 wt. %; from about 40 to about 60 wt. %, about 40 to about 55 wt. %, about 40 to about 50 wt. %, about 40 to about 45 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). The polyol may be a humectant. In some preferred embodiments, the polyol(s) comprises glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition comprises maltitol, mannitol, sorbitol, xylitol, a polypropylene glycol (PPG), a polyethylene glycol (PEG), a block copolymer of PPG and PEG, or a combination or two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be included in the oral care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture of two or more thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600 (CAS-25322-68-3). In some embodiments, the oral care compositions of the disclosure comprise one or more polyethylene glycols, for example, polyethylene glycols in a molecular weight range from 200 to 800. For example, the oral care compositions may comprise one or more of polyethylene glycol 200, polyethylene glycol 300, polyethylene glycol 400, polyethylene glycol, 600 or polyethylene glycol 800.

The oral care compositions may, in some cases, comprise one or more amino acid(s) in an effective amount. In some embodiments, the one or more amino acid(s) may be present in the oral care composition in an amount from about 0.1 to about 10 wt. %, about 0.1 to about 8 wt. %, about 0.1 to about 6 wt. %, about 0.1 to about 4 wt. %, about 0.1 to about 2 wt. %, about 0.1 to about 1 wt. %, about 0.1 to about 0.5 wt. %; from about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %; from about 6 to about 10 wt. %, about 6 to about 8 wt. %; from about 8 to about 10 wt. %, including any range or subrange thereof, based on the total weight of the oral care composition.

The one or more amino acids may be selected from basic amino acids, neutral amino acids, and combinations thereof. The basic amino acids may be selected from naturally occurring basic amino acids, such as arginine, lysine, and histidine, and non-naturally occurring basic amino acids having a carboxyl group and an amino group in the molecule, which are water-soluble and provide an aqueous solution with a pH of 7 or greater. Examples of basic amino acids include arginine, lysine, serine, citrulline, ornithine, creatine, histidine, diaminobutanoic acid, diaminoproprionic acid, salts thereof or combinations thereof. In some embodiments, the basic amino acids are selected from arginine, citrulline, and ornithine. In certain embodiments, the basic amino acid is arginine, e.g., L-arginine, or a salt thereof. Additionally or alternatively, the one or more amino acid can be chosen from neutral amino acids, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

In some embodiments, the oral care compositions may include one or more sweeteners. The oral care composition may include caloric sweeteners and/or non-caloric sweeteners. Examples of non-caloric sweeteners include saccharin, for example, sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 50 wt. %, based on the total weight of the oral care composition. For example, the oral care composition may have a total amount of sweetener(s) from about 0.1 to about 40 wt. %, about 0.1 to about 30 wt. %, about 0.1 to about 20 wt. %, about 0.1 to about 10 wt. %, about 0.1 to about 5 wt. %, about 0.1 to about 3 wt. %; from about 1 to about 50 wt. %, about 1 to about 40 wt. %, about 1 to about 30 wt. %, about 1 to about 20 wt. %, about 1 to about 10 wt. %, about 1 to about 5 wt. %, about 1 to about 3 wt. %; from about 5 to about 50 wt. %, about 5 to about 40 wt. %, about 5 to about 30 wt. %, about 5 to about 20 wt. %, about 5 to about 10 wt. %; from about 10 to about 50 wt. %, about 10 to about 45 wt. %, about 10 to about 40 wt. %, about 10 to about 35 wt. %, about 10 to about 30 wt. %, about 10 to about 25 wt. %; from about 15 to about 50 wt. %, about 15 to about 45 wt. %, about 15 to about 40 wt. %, about 15 to about 37 wt. %, about 15 to about 34 wt. %, about 15 to about 31 wt. %, about 15 to about 28 wt. %, about 15 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 50 wt. %, about 37 to about 45 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, and various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more pigments, such as whitening pigments, and/or colorants. In some embodiments, the whitening pigments include particles ranging in size from about 0.1 μm to about 10 μm with a refractive index greater than about 1.2. Suitable whitening agents include, without limitation, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof.

Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

The oral care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween.

The oral care compositions may include water in some embodiments. The water may be present in the oral care composition in an amount from about 5 to about 37 wt. %, based on the total weight of the oral care composition. For example, the oral care composition may include water in an amount from about 5 to about 34 wt. %, about 5 to about 31 wt. %, about 5 to about 28 wt. %, about 5 to about 25 wt. %, about 5 to about 20 wt. %; from about 10 to about 37 wt. %, about 10 to about 34 wt. %, about 10 to about 31 wt. %, about 10 to about 28 wt. %, about 10 to about 25 wt. %; from about 15 to about 37 wt. %, about 15 to about 34 wt. %, about 15 to about 31 wt. %, about 15 to about 28 wt. %, about 15 to about 25 wt. %; from about 20 to about 37 wt. %, about 20 to about 34 wt. %, about 20 to about 31 wt. %, about 20 to about 28 wt. %; from about 25 to about 37 wt. %, about 25 to about 34 wt. %, about 25 to about 31 wt. %; from about 28 to about 37 wt. %, about 28 to about 34 wt. %, about 28 to about 31 wt. %; from about 31 to about 37 wt. %; from about 37 to about 50 wt. %, about 37 to about 45 wt. %, or any range or subrange thereof, based on the total weight of the oral care composition.

In another aspect, the present disclosure provides a method of treatment or prevention of erosive tooth demineralization, repair of enamel, gingivitis, plaque, and/or dental caries, the method comprising the application to the oral cavity of a subject in need thereof an oral composition disclosed herein, e.g., by brushing, for example, one or more times per day. The methods may treat, reduce or control the incidence of enamel erosion.

The methods typically comprise applying any of the compositions as described herein to the teeth, e.g., by brushing, rubbing, swishing when the oral care composition is in the form of a mouthwash, or otherwise administering the oral care compositions to the oral cavity of a subject. The oral care compositions can be administered regularly, such as, for example, one or more times per day. In various embodiments, administering the oral care compositions of the present disclosure to a subject can provide one or more of the following benefits: (i) reduce hypersensitivity of the teeth, (ii) reduce plaque accumulation, (iii) reduce or inhibit demineralization and promote remineralization of the teeth, (iv) inhibit microbial biofilm formation in the oral cavity, (v) reduce or inhibit gingivitis, (vi) promote healing of sores or cuts in the mouth, (vii) reduce levels of acid producing bacteria, (viii) increase relative levels of non-cariogenic and/or non-plaque forming bacteria, (ix) reduce or inhibit formation of dental caries, (x) reduce, repair or inhibit pre-carious lesions of the enamel, e.g., as detected by quantitative light-induced fluorescence (QLF) or electrical caries measurement (ECM), (xi) treat, relieve or reduce dry mouth, (xii) clean the teeth and oral cavity, (xiii) reduce erosion, (xiv) whiten teeth; (xv) reduce tartar build-up, and/or (xvi) promote systemic health, including cardiovascular health, e.g., by reducing potential for systemic infection via the oral tissues. The disclosure further provides compositions for use in any of the above methods. Further embodiments provide methods wherein at least one tooth is remineralized after administration of an oral composition as described herein.

### EXAMPLES

### Example 1

Two non-limiting examples of oral care composition (Example Compositions A and B) were prepared in the form of dentifrices in accordance with aspects of the invention. Five comparative compositions (Comparative Compositions 1-5) were also prepared in the form of dentifrices. Example Compositions A and B and Comparative Compositions 1-5 were produced in accordance with the formulations shown in Table 1.

TABLE 1

| | Ex. A | Comp. 1 | Comp. 2 | Ex. B | Comp. 3 | Comp. 4 | Comp. 5 |
|---|---|---|---|---|---|---|---|
| US INCI Name | (Wt. %) | (Wt. %) | (Wt. %) | (Wt. %) | (Wt. %) | (Wt. %) | (Wt. %) |
| Sorbitol | 43 | 43 | 43 | 43 | 43 | 43 | 43 |
| Polyethylene Glycol | 2 | 2 | 2 | 2 | 2 | 2 | 2 |
| Synthetic Amorphous | 15 | 15 | 15 | 15 | 15 | 15 | 15 |
| Silica | | | | | | | |
| Synthetic Thickening | 8 | 8 | 8 | 8 | 8 | 8 | 8 |
| Silica | | | | | | | |
| Xanthan Gum | 0.4 | 0.4 | 0.4 | 0.4 | 0.4 | 0.4 | 0.4 |
| Sodium Methyl Cocoyl | 1.4 | | 2.8 | | | | 1.4 |
| Taurate | | | | | | | |
| Cocamidopropyl | 0.4 | 0.8 | | | | | |
| Betaine | | | | | | | |
| Lauryl Glucoside | | | | 0.7 | 1.4 | | |
| Sodium Cocoyl | | | | 0.7 | | 1.4 | 0.6 |
| Glutamate | | | | | | | |
| Stannous Fluoride | 0.5 | 0.5 | 0.5 | 0.5 | 0.5 | 0.5 | 0.5 |
| Potassium Nitrate | 0.5 | 0.5 | 0.5 | 0.5 | 0.5 | 0.5 | 0.5 |
| Tetrasodium | 1.2 | 1.2 | 1.2 | 1.2 | 1.2 | 1.2 | 1.2 |
| Pyrophosphate | | | | | | | |
| Flavoring Agents, | 2 | 2 | 2 | 2 | 2 | 2 | 2 |
| Sweeteners, and | | | | | | | |
| Coloring Agents | | | | | | | |
| Water | QS to | QS to | QS to | QS to | QS to | QS to | QS to |
| | 100 | 100 | 100 | 100 | 100 | 100 | 100 |

### Example 2

Example Compositions A and B and Comparative Compositions 1-5 were evaluated to assess the foaming characteristics of such dentifrices. Specifically, the foaming characteristics were evaluated using a SITA foam tester. The SITA foam tester provides automated agitation of diluted toothpaste preparations to mimic the mechanical action experienced during brushing, which correlates with sensory ratings by expert (trained) panelists. The SITA foam tester quantifies foam generation and decay rate of the foam by utilizing an array of needles that monitor changes in electro-conductance. The change in foam height is reported by the instrument in ten second intervals up to 120 seconds after a pre-determined amount of agitation has been applied to the test solution. Similarly, foam decay is measured in ten second intervals up to one minute but with no further agitation of the solution. The results shown in Table 2 are reported for the average foam height after 3 separate runs measured during foam generation at 30, 60, and 120 seconds of agitation and foam height after one minute of decay with agitation removed.

A summary of the foam characteristics for Example Compositions A and B and Comparative Compositions 1-5 are shown in Table 2.

TABLE 2

| | 30 sec foam vol. | 60 sec foam vol. | 120 sec foam vol. |
|---|---|---|---|
| | (mL) | (mL) | (mL) |
| Ex. A | 119 | 147 | 184 |
| Comp. 1 | 103 | 128 | 168 |
| Comp. 2 | 151 | 181 | 220 |
| Ex. B | 106 | 136 | 164 |
| Comp. 3 | 6 | 2 | 4 |
| Comp. 4 | 72 | 81 | 94 |
| Comp. 5 | 58 | 70 | 95 |

Example Composition B achieved a significant improvement in foaming volume as compared to Comparative Compositions 3 and 4. The significant improvement of the foaming characteristics achieved by Example Composition B was unexpected as Comparative Compositions 3 and 4 used surfactants present in Example Composition B and had similar total amounts of surfactants. Additionally, Comparative Composition 5, which included a total amount of surfactants that was greater than Example Compositions A and B produced significantly less foaming volume than either Example Composition A or B. Without being limited to any particular theory, it is believed that Example Composition B exhibits a synergistic effect with regard to foaming characteristics.

### Example 3

Example Compositions A and B and Comparative Compositions 1-5 were further evaluated to assess their respective characteristics. During the evaluation of Example Compositions A and B and Comparative Compositions 1-5, it was noted that Comparative Composition 2 had a particularly opaque appearance. The inventors did not expect Example Composition A to achieve the level of foam exhibited while not having a particularly opaque appearance in view of the Comparative Compositions 1 and 2.

### Example 4

Two non-limiting examples of oral care composition (Example Compositions C and D) were prepared in the form of dentifrices in accordance with aspects of the invention. Example Compositions C and D were produced in accordance with the formulations shown in Table 3.

TABLE 3

| US INCI Name | Ex. C (Wt. %) | Ex. D (Wt. %) |
|---|---|---|
| Sorbitol | 37 | 42 |
| Polyethylene Glycol | 2 | 2 |
| 600 | | |
| Synthetic Amorphous | 15 | 15 |
| Silica | | |
| Synthetic Thickening | 10 | 7 |
| Silica | | |
| Xanthan Gum | 0.4 | 0.4 |
| Sodium Methyl Cocoyl | | 2.5 |
| Taurate | | |
| Cocamidopropyl | 0.9 | 0.9 |
| Betaine | | |
| Lauryl Glucoside | 1 | |
| Sodium Cocoyl | 1 | |
| Glutamate | | |
| Stannous Fluoride | 0.5 | 0.5 |
| Potassium Nitrate | 0.5 | 0.5 |
| Tetrasodium | 1.2 | 1.2 |
| Pyrophosphate | | |
| Flavoring Agents, | 2 | 2 |
| Sweeteners, and | | |
| Coloring Agents | | |
| Water | QS to 100 | QS to 100 |

1. An oral care composition comprising:
a surfactant system comprising at least one of:
a non-sulfate based anionic surfactant and an amphoteric surfactant, or
a nonionic surfactant and an amino acid surfactant;
a stannous ion source;
a nitrate ion source; and
a phosphate source.

2. The oral care composition according to claim 1, wherein the non-sulfate based anionic surfactant is selected from sulfonated monoglycerides of fatty acids, acyl isethionates, acyl sarcosinates, acyl taurates, and a combination of two or more thereof.

3. The oral care composition according to claim 1, wherein the non-sulfate based anionic surfactant comprises an acyl taurate.

4. (canceled)

5. The oral care composition according to claim 1, wherein the non-sulfate based anionic surfactant comprises sodium methyl cocoyl taurate.

6. The oral care composition according to claim 1, wherein the amphoteric surfactant is selected from alkyl amphopropionates, betaines, alkyl sultaines, alkyl amphoacetates, and a combination of two or more thereof.

7. The oral care composition according to claim 1, wherein the amphoteric surfactant comprises a betaine surfactant.

8. (canceled)

9. The oral care composition according to claim 1, wherein the surfactant system is present in an amount from about 0.5 to about 9 wt. %, based on the total weight of the oral care composition.

10. The oral care composition according to claim 1, wherein the non-sulfate based anionic surfactant is present in an amount from about 0.1 to about 7 wt. %, based on the total weight of the oral care composition.

11. The oral care composition according to claim 1, wherein the amphoteric surfactant is present in an amount from about 0.1 to about 5 wt. %, based on the total weight of the oral care composition.

12. The oral care composition according to claim 1, wherein the oral care composition is substantially free of or free of sodium lauryl sulfate and sodium lauryl ether sulfate.

13. The oral care composition according to claim 1, wherein the nonionic surfactant comprises a glucoside surfactant.

14. The oral care composition according to claim 13, wherein the glucoside surfactant is selected from lauryl glucoside, lauroyl ethyl glucoside, myristoyl ethyl glucoside, oleoyl ethyl glucoside, and a combination of two or more thereof.

15. The oral care composition according to claim 1, wherein the amino acid derived surfactant is a glutamic acid derived surfactant having an aliphatic carbon chain of 3 to 21 carbons.

16. (canceled)

17. (canceled)

18. The oral care composition according to claim 1, wherein the amino acid derived surfactant comprises sodium cocoyl glutamate.

19. The oral care composition according to claim 1, wherein the nonionic surfactant is present in an amount from about 0.1 to about 7 wt. %, based on the total weight of the oral care composition.

20. The oral care composition according to claim 1, wherein the amino acid derived surfactant is present in an amount from about 0.1 to about 7 wt. %, based on the total weight of the oral care composition.

21. The oral care composition according to claim 1, wherein the oral care composition has a weight ratio of the total amount of anionic surfactant(s) to the total amount of amphoteric surfactant(s) of from about 1:1 to about 5:1.

22. The oral care composition according to claim 1, wherein the oral care composition has a weight ratio of the total amount of amino acid based surfactant(s) to the total amount of nonionic surfactant(s) of from about 1:4 to about 4:1.

23. An oral care composition comprising:
a surfactant system comprising:
a betaine surfactant, and
a taurate surfactant;
a stannous ion source;
a nitrate ion source; and
a phosphate source.

24. An oral care composition comprising:
a surfactant system comprising:
a glutamate surfactant, and
a glucoside surfactant;
a stannous ion source;
a nitrate ion source; and
a phosphate source.
